(19) United States
(12) Patent Application Publication
(10) Pub. No.: US 2022/0234010 A1
(43) Pub. Date: Jul. 28, 2022

(54) AUTOMATED RECYCLED CLOSED-LOOP WATER BASED DRILLING FLUID CONDITION MONITORING SYSTEM

(71) Applicant: SAUDI ARABIAN OIL COMPANY, Dhahran (SA)

(72) Inventors: Mahmoud F. Abughaban, Dhahran (SA); Ashok Kumar Santra, The Woodlands, TX (US)

(73) Assignees: SAUDI ARABIAN OIL COMPANY, Dhahran (SA); ARAMCO SERVICES COMPANY, Houston, TX (US)

(21) Appl. No.: 17/157,290

(22) Filed: Jan. 25, 2021

(51) Int. Cl. B01F 15/00; E21B 21/06; B01F 3/08; B01F 3/12; G01N 33/28; G01N 11/00; G01N 9/00

(52) U.S. Cl. CPC B01F 15/00253; B01F 2215/0081; B01F 3/0803; B01F 3/088; B01F 3/1207; B01F 3/1271; B01F 15/00188; B01F 15/0022; B01F 15/00246; B01F 15/00292; B01F 15/00233; B01F 15/00344; G01N 33/2823; G01N 33/2847; G01N 11/00; G01N 9/00; E21B 21/062

(57) ABSTRACT

A closed-loop drilling fluid condition system for drilling fluid mixing and recycling process is described. The closed-loop drilling fluid condition system improves drilling fluid quality, repeatability, utilization efficiency, and health, safety and environment (HSE) issues. In particular, the closed-loop drilling fluid condition system automates a water-based drilling fluid workflow or an oil-based drilling fluid workflow where individual stages are monitored and adjusted in real-time. Specifically, the individual stages are monitored in real-time using sensors and adjusted in real-time based on commands from a monitoring device to achieve specific drilling fluid parameters.

### BACKGROUND

Drilling fluid, also referred to as “drilling mud” or simply “mud,” is used to facilitate drilling boreholes into the earth, such as drilling oil and natural gas wells. Three main categories of drilling fluids include dispersed or non-dispersed water-based drilling fluids (WBs), non-aqueous drilling fluids usually referred to as oil-based drilling fluids (OBs), and gaseous drilling fluids. Appropriate polymer and clay additives are often added in these drilling fluids.

The main functions of drilling fluids include providing hydrostatic pressure to prevent formation fluids from entering into the borehole, keeping the drill bit cool and clean during drilling, carrying out drill cuttings, and suspending the drill cuttings while drilling is paused and when the drilling assembly is brought in and out of the borehole.

### SUMMARY

In general, in one aspect, the invention relates to a method for drilling fluid mixing and recycling. The method includes receiving, by a human monitor interface (HMI) from a user, specified drilling fluid properties, adding, in response to a first control signal from the HMI to control a first dispensing device, a volume of water in a mixing tank to reach a volume threshold, adding, in response to a second control signal from the HMI to control a second dispensing device, a viscosifer in the mixing tank to reach a viscosity threshold, adding, in response to a fourth control signal from the HMI to control a fourth dispensing device, a weighting agent in the mixing tank to reach a specific gravity threshold, adding, in response to a fifth control signal from the HMI to control a fifth dispensing device, a pH buffer solution in the mixing tank to reach a pH threshold, and releasing, in response to a sixth control signal from the HMI to control a sixth dispensing device, a final drilling fluid from the mixing tank to a drill string for continuous drilling, wherein the volume threshold, the viscosity threshold, the specific gravity threshold, and the pH threshold are determined based on the specified drilling fluid properties.

In general, in one aspect, the invention relates to a system for drilling fluid mixing and recycling. The system includes a plurality of real-time drilling fluid properties sensors configured to measure real-time fluid properties in a mixing tank, a human monitor interface (HMI) configured to receiving a user input of specified drilling fluid properties, receiving, from the plurality of real-time drilling fluid properties sensors; sensor outputs representing the real-time fluid properties in the mixing tank, and generate, based on the user input and the sensor outputs, a sequence of control signals for drilling fluid mixing and recycling, an automated material transfer unit configured to transfer chemical ingredients to an automated drilling fluid processing system, and the automated drilling fluid processing system configured to add, in response to a first control signal from the HMI to control a first dispensing device, a volume of water in the mixing tank to reach a volume threshold, add, in response to a second control signal from the HMI to control a second dispensing device, a viscosifer in the mixing tank to reach a viscosity threshold, add, in response to a fourth control signal from the HMI to control a fourth dispensing device, a weighting agent in the mixing tank to reach a specific gravity threshold, add, in response to a fifth control signal from the HMI to control a fifth dispensing device, a pH buffer solution in the mixing tank to reach a pH threshold, and release, in response to a sixth control signal from the HMI to control a sixth dispensing device, a final drilling fluid from the mixing tank to a drill string for continuous drilling, wherein the volume threshold, the viscosity threshold, the specific gravity threshold, and the pH threshold are determined based on the specified drilling fluid properties.

In general, in one aspect, the invention relates to a non-transitory computer readable medium storing instructions executable by a computer processor for drilling fluid mixing and recycling. The instructions, when executed, include functionality for receiving, by a human monitor interface (HMI) from a user, specified drilling fluid properties, adding, in response to a first control signal from the HMI to control a first dispensing device, a volume of water in a mixing tank to reach a volume threshold, adding, in response to a second control signal from the HMI to control a second dispensing device, a viscosifer in the mixing tank to reach a viscosity threshold, adding, in response to a fourth control signal from the HMI to control a fourth dispensing device, a weighting agent in the mixing tank to reach a specific gravity threshold, adding, in response to a fifth control signal from the HMI to control a fifth dispensing device, a pH buffer solution in the mixing tank to reach a pH threshold, and releasing, in response to a sixth control signal from the HMI to control a sixth dispensing device, a final drilling fluid from the mixing tank to a drill string for continuous drilling, wherein the volume threshold, the viscosity threshold, the specific gravity threshold, and the pH threshold are determined based on the specified drilling fluid properties.

Other aspects and advantages will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIGS. 1 and 2 show systems in accordance with one or more embodiments.

FIG. 3 shows a flowchart in accordance with one or more embodiments.

FIGS. 4A and 4B show a computing system in accordance with one or more embodiments.

### DETAILED DESCRIPTION

Specific embodiments of the disclosure will now be described in detail with reference to the accompanying figures. Like elements in the various figures are denoted by like reference numerals for consistency.

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

Embodiments of the invention provide a method, a system, and a non-transitory computer readable medium for drilling fluid mixing and recycling process by creating a closed-loop drilling fluid condition system that improves drilling fluid quality, repeatability, utilization efficiency, and health, safety and environment (HSE) issues. In one or more embodiments of the invention, the closed-loop drilling fluid condition system automates a water-based drilling fluid workflow or an oil-based drilling fluid workflow where individual stages are monitored and adjusted in real-time. Specifically, the individual stages are monitored in real-time using sensors and adjusted in real-time based on commands from a monitoring device to achieve specific drilling fluid parameters.

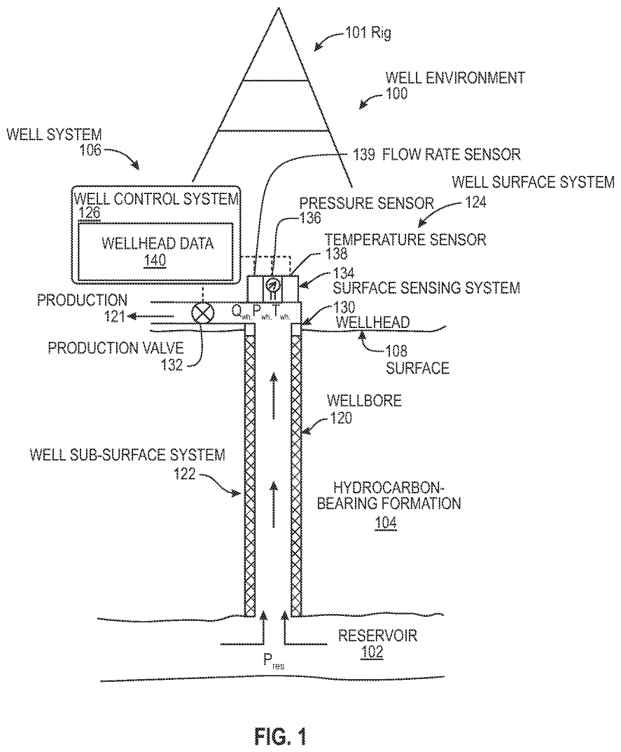

Turning to FIG. 1, FIG. 1 shows a schematic diagram in accordance with one or more embodiments. As shown in FIG. 1, FIG. 1 illustrates a well environment (100) that includes a hydrocarbon reservoir (“reservoir”) (102) located in a subsurface hydrocarbon-bearing formation (“formation”) (104) and a well system (106). The hydrocarbon-bearing formation (104) may include a porous or fractured rock formation that resides underground, beneath the earth's surface (“surface”) (108). In the case of the well system (106) being a hydrocarbon well, the reservoir (102) may include a portion of the hydrocarbon-bearing formation (104). The hydrocarbon-bearing formation (104) and the reservoir (102) may include different layers of rock having varying characteristics, such as varying degrees of permeability, porosity, capillary pressure, and resistivity. In the case of the well system (106) being operated as a production well, the well system (106) may facilitate the extraction of hydrocarbons (or “production”) from the reservoir (102).

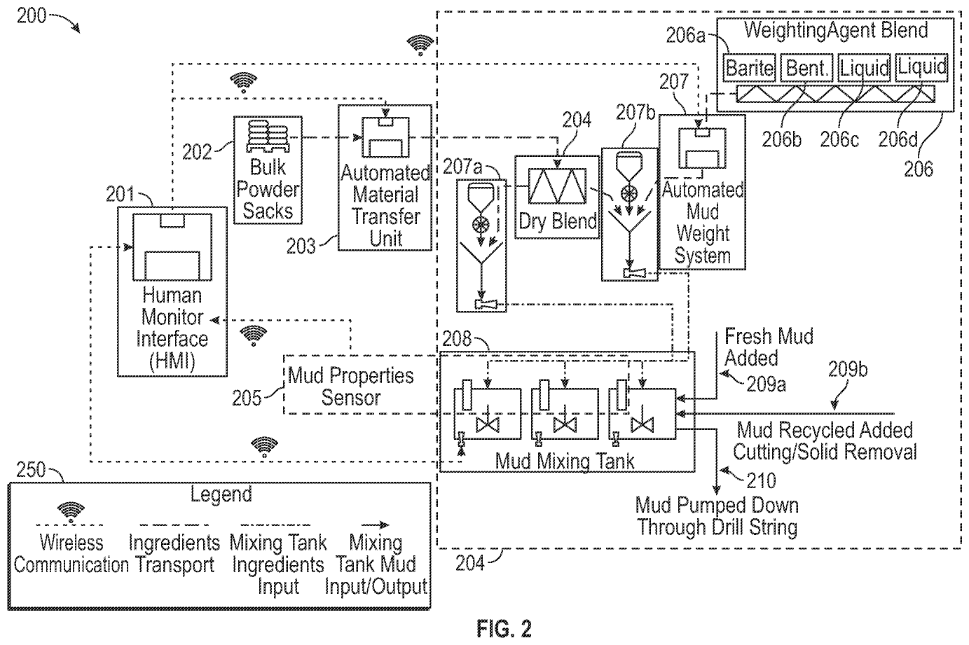

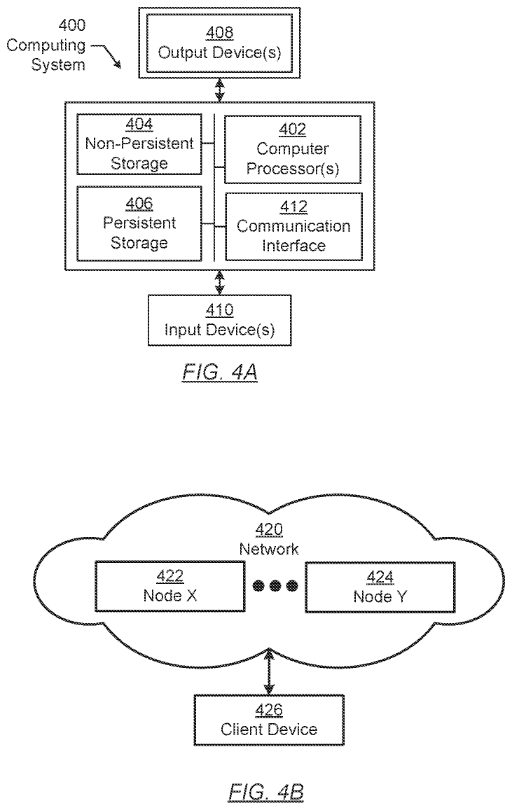

In some embodiments of the invention, the well system (106) includes a rig (101), a wellbore (120), a well sub-surface system (122), a well surface system (124), and a well control system (“control system”) (126). The control system (126) may control various operations of the well system (106), such as well production operations, well drilling operation, well completion operations, well maintenance operations, and reservoir monitoring, assessment and development operations. In some embodiments, the control system (126) includes a computer system that is the same as or similar to that of computer system (400) described below in FIGS. 4A and 4B and the accompanying description. For example, the human monitor interface (HMI) (201) depicted in FIG. 2 below may be part of the control system (126) and may be implemented as a combination of hardware and software components of the computer system (400).

The rig (101) is the machine used to drill a borehole to form the wellbore (120). Major components of the rig (101) include the drilling fluid tanks, the drilling fluid pumps (e.g., rig mixing pumps), the derrick or mast, the drawworks, the rotary table or topdrive, the drillstring, the power generation equipment and auxiliary equipment. In one or more embodiments of the invention, the drilling fluid is monitored and adjusted in real-time using the closed-loop drilling fluid condition system (200) described in reference to FIG. 2 below.

The wellbore (120) includes a bored hole (i.e., borehole) that extends from the surface (108) into a target zone of the hydrocarbon-bearing formation (104), such as the reservoir (102). An upper end of the wellbore (120), terminating at or near the surface (108), may be referred to as the “up-hole” end of the wellbore (120), and a lower end of the wellbore, terminating in the hydrocarbon-bearing formation (104), may be referred to as the “downhole” end of the wellbore (120). The wellbore (120) may facilitate the circulation of drilling fluids during drilling operations, the flow of hydrocarbon production (“production”) (121) (e.g., oil and gas) from the reservoir (102) to the surface (108) during production operations, the injection of substances (e.g., water) into the hydrocarbon-bearing formation (104) or the reservoir (102) during injection operations, or the communication of monitoring devices (e.g., logging tools) into the hydrocarbon-bearing formation (104) or the reservoir (102) during monitoring operations (e.g., during in situ logging operations).

In some embodiments, during operation of the well system (106), the control system (126) collects and records wellhead data (140) for the well system (106). The wellhead data (140) may include, for example, a record of measurements of wellhead pressure (P_{wh}) (e.g., including flowing wellhead pressure), wellhead temperature (T_{wh}) (e.g., including flowing wellhead temperature), wellhead production rate (Q_{wh}) over some or all of the life of the well (106), and water cut data. The wellhead data (140) may also include sensor data of the closed-loop drilling fluid condition system (200) depicted in FIG. 2 below. In some embodiments, the measurements are recorded in real-time, and are available for review or use within seconds, minutes or hours of the condition being sensed (e.g., the measurements are available within 1 hour of the condition being sensed). In such an embodiment, the wellhead data (140) may be referred to as “real-time” wellhead data (140). Real-time wellhead data (140) may enable an operator of the well (106) to assess a relatively current state of the well system (106), and make real-time decisions regarding development of the well system (106) and the reservoir (102), such as on-demand adjustments in drilling fluid and regulation of production flow from the well.

In some embodiments, the well sub-surface system (122) includes casing installed in the wellbore (120). For example, the wellbore (120) may have a cased portion and an uncased (or “open-hole”) portion. The cased portion may include a portion of the wellbore having casing (e.g., casing pipe and casing cement) disposed therein. The uncased portion may include a portion of the wellbore not having casing disposed therein. In some embodiments, the casing includes an annular casing that lines the wall of the wellbore (120) to define a central passage that provides a conduit for the transport of tools and substances through the wellbore (120). For example, the central passage may provide a conduit for lowering logging tools into the wellbore (120), a conduit for the flow of production (121) (e.g., oil and gas) from the reservoir (102) to the surface (108), or a conduit for the flow of injection substances (e.g., water) from the surface (108) into the hydrocarbon-bearing formation (104). In some embodiments, the well sub-surface system (122) includes production tubing installed in the wellbore (120). The production tubing may provide a conduit for the transport of tools and substances through the wellbore (120). The production tubing may, for example, be disposed inside casing. In such an embodiment, the production tubing may provide a conduit for some or all of the production (121) (e.g., oil and gas) passing through the wellbore (120) and the casing.

In some embodiments, the well surface system (124) includes a wellhead (130). The wellhead (130) may include a rigid structure installed at the “up-hole” end of the wellbore (120), at or near where the wellbore (120) terminates at the Earth's surface (108). The wellhead (130) may include structures for supporting (or “hanging”) casing and production tubing extending into the wellbore (120). Production (121) may flow through the wellhead (130), after exiting the wellbore (120) and the well sub-surface system (122), including, for example, the casing and the production tubing. In some embodiments, the well surface system (124) includes flow regulating devices that are operable to control the flow of substances into and out of the wellbore (120). For example, the well surface system (124) may include one or more production valves (132) that are operable to control the flow of production (134). For example, a production valve (132) may be fully opened to enable unrestricted flow of production (121) from the wellbore (120), the production valve (132) may be partially opened to partially restrict (or “throttle”) the flow of production (121) from the wellbore (120), and production valve (132) may be fully closed to fully restrict (or “block”) the flow of production (121) from the wellbore (120), and through the well surface system (124).

In some embodiments, the wellhead (130) includes a choke assembly. For example, the choke assembly may include hardware with functionality for opening and closing the fluid flow through pipes in the well system (106). Likewise, the choke assembly may include a pipe manifold that may lower the pressure of fluid traversing the wellhead. As such, the choke assembly may include set of high pressure valves and at least two chokes. These chokes may be fixed or adjustable or a mix of both. Redundancy may be provided so that if one choke has to be taken out of service, the flow can be directed through another choke. In some embodiments, pressure valves and chokes are communicatively coupled to the well control system (126). Accordingly, a well control system (126) may obtain wellhead data regarding the choke assembly as well as transmit one or more commands to components within the choke assembly in order to adjust one or more choke assembly parameters.

Keeping with FIG. 1, in some embodiments, the well surface system (124) includes a surface sensing system (134). The surface sensing system (134) may include sensors for sensing characteristics of substances, including production (121), passing through or otherwise located in the well surface system (124). The characteristics may include, for example, pressure, temperature and flow rate of production (121) flowing through the wellhead (130), or other conduits of the well surface system (124), after exiting the wellbore (120). The surface sensing system (134) may also include sensors for sensing characteristics of the rig (101), such as bit depth, hole depth, drilling fluidflow, hook load, rotary speed, etc.

In some embodiments, the surface sensing system (134) includes a surface pressure sensor (136) operable to sense the pressure of production (151) flowing through the well surface system (124), after it exits the wellbore (120). The surface pressure sensor (136) may include, for example, a wellhead pressure sensor that senses a pressure of production (121) flowing through or otherwise located in the wellhead (130). In some embodiments, the surface sensing system (134) includes a surface temperature sensor (138) operable to sense the temperature of production (151) flowing through the well surface system (124), after it exits the wellbore (120). The surface temperature sensor (138) may include, for example, a wellhead temperature sensor that senses a temperature of production (121) flowing through or otherwise located in the wellhead (130), referred to as “wellhead temperature” (T_{wh}). In some embodiments, the surface sensing system (134) includes a flow rate sensor (139) operable to sense the flow rate of production (151) flowing through the well surface system (124), after it exits the wellbore (120). The flow rate sensor (139) may include hardware that senses a flow rate of production (121) (Q_{wh}) passing through the wellhead (130). In some embodiments, the surface sensing system (134) includes the real-time drilling fluid properties sensors (205) depicted in FIG. 2 below.

Turning to FIG. 2, FIG. 2 illustrates an example closed-loop drilling fluid condition system in accordance with one or more embodiments of the invention. In one or more embodiments, one or more of the modules and/or elements shown in FIG. 2 may be omitted, repeated, and/or substituted. Accordingly, embodiments of the invention should not be considered limited to the specific arrangements of modules and/or elements shown in FIG. 2.

In particular, FIG. 2 shows a closed-loop drilling fluid condition system (200) for drilling fluid mixing and recycling process. In one or more embodiments of the invention, the closed-loop drilling fluid condition system (200) provides automatic recycling and other drilling fluid processing operation at the rig (101) in the well environment (100) depicted in FIG. 1 above. The closed-loop drilling fluid condition system (200) automatically removes cutting/solids from the used drilling fluid, making up volume and re-adjusting water based drilling fluids properties (e.g., rheological behavior, pH, density, etc.) using drilling fluid additives before circulating drilling back through the drill string for continuous drilling. As such, the closed-loop drilling fluid condition system (200) automates drilling fluid recycling and treatment process and improves the HSE issues, increase efficiency, improves drilling fluid quality, reduces loss of net productive time (NPT), and provide the ability to create a process.

As shown in FIG. 2, the closed-loop drilling fluid condition system (200) includes four main components, namely: a human monitor interface (HMI) (201), an automated material transfer unit (203), an automated drilling fluid processing system (204), and real-time drilling fluid properties sensors (205). The human monitor interface (HMI) (201) is an electronic device that includes a combination of hardware and software components. The automated material transfer unit (203) and automated drilling fluid processing system (204) are electro-mechanical devices that include also a combination of hardware and software components. In particular, the automated material transfer unit (203) and automated drilling fluid processing system (204) include mechanical and robotic mechanisms (e.g., conveyor belt, robotic arm, pipes, valves, choke, etc.) that are actuated in response to wired and/or wireless control signals from the HMI (201). The material transfer mechanism and wired/wireless communication between the main components are depicted according to the legend (250). The material input to the closed-loop drilling fluid condition system (200) includes the bulk powder sacks (202), the weighting agent blend (206), and the recycled drilling fluid (209b) with cutting/solids removed. The bulk powder sacks (202) is a storage device with automated dispensing mechanism to dispense chemical ingredients in dry powder form. The weighting agent blend (206) is a storage device with automated dispensing mechanism to dispense chemicals in liquid form. The material output from the closed-loop drilling fluid condition system (200) is the final drilling fluid (210) circulating back through the drill string for continuous drilling, as indicated by the arrow (210).

The human monitor interface (HMI) (201) is a device located at the rig site (e.g., rig (101) depicted in FIG. 1) that receives user input to specify drilling fluid properties, weight, and volume. To prepare fresh drilling fluid initially before any recycling, HMI (201) activates the automated material transfer unit (203) to supply material from the bulk powder sacks (202) into the dry-blend (204) at a specified weight and volume to the selected mixing tank (208) where a required amount of water (206c) is metered through a flow meter. Dry blend (204) is a storage device for preparing the drilling fluid from chemical ingredients in the dry powder form without using recycled drilling fluid containing ingredients in the “wet” form. As directed by the HMI (204), chemical ingredients from the dry blend (204) is selectively mixed and transferred via the rig mixing pump (207a) to the mixing tank (208). Further directed by the HMI (204), chemical ingredients from the dry blend (204) and weighting agent blend (206) are selectively mixed and transferred via the rig mixing pump (207b) to the mixing tank (208). The mixing and transferring of the chemical ingredients continue until outputs from the real-time drilling fluid properties sensors (205) indicate that desired drilling fluid properties (e.g., rheological behavior, pH, density, etc.) are obtained. In particular, the outputs from the real-time drilling fluid properties sensors (205) are monitored in real-time by the HMI (201) to determine when the desired drilling fluid properties are obtained. Specifically, the sensor outputs are transmitted to the HMI (201) and final adjustment of drilling fluids properties is performed until the user specified drilling fluid properties are achieved.

The drilling fluid may also be prepared including the recycled drilling fluid (209b) after removing cutting/solids and fine tuning the rheological parameters by way of controlled addition of liquid (diluted) version of various rheology modifiers. Additional additives such as pH buffer, H₂S/CO₂ scavenger, oxygen scavenger and shale inhibitor may also be added at this stage. For example, the real-time drilling fluid properties sensors (205) may include a pH sensor, density sensor, rheology sensor, etc. to fine tune the drilling fluid parameters as described above.

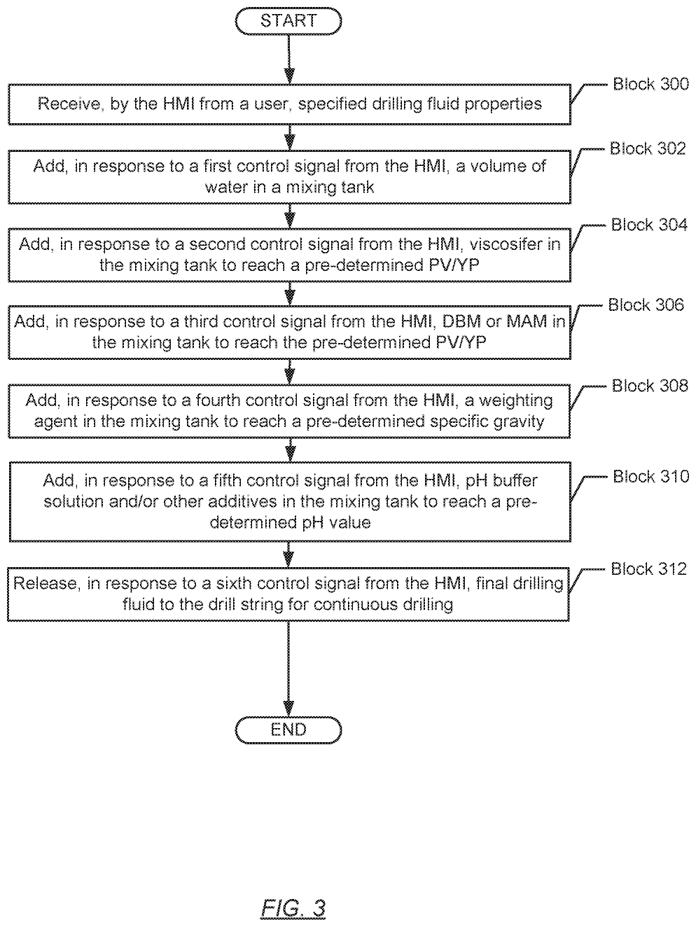

Turning to FIG. 3, FIG. 3 shows a method flowchart in accordance with one or more embodiments. Specifically, FIG. 3 describes a method of real-time drilling fluid mixing procedure. One or more blocks in FIG. 3 may be performed using one or more components as described in FIGS. 1 and 2. While the various blocks in FIG. 3 are presented and described sequentially, one of ordinary skill in the art will appreciate that some or all of the blocks may be executed in different orders, may be combined or omitted, and some or all of the blocks may be executed in parallel and/or iteratively. Furthermore, the blocks may be performed actively or passively.

Initially in Block 300, specified drilling properties are received by the HMI (201) from a user. The specified drilling properties include one or more of weight, volume, viscosity, yield point, specific gravity, rheological behavior, pH, and density of the final drilling fluid. Subsequent to receiving the specified drilling fluid properties, a command is received by the HMI (201) from the user to initiate the real-time drilling fluid mixing procedure. In response to the command, the HMI (201) generates a sequence of control signals (i.e., the first, second, third, fourth, fifth, and sixth control signals) to perform the method blocks described below.

In Block 302, in response to a first control signal from the HMI (201), a specified volume of water is added to the mixing tank (208). The specified volume is determined based on the target volume of the final drilling fluid specified by the user to the HMI (201). The first control signal actuates a first valve controlling the weighting agent blend (206) to allow water (206c) to flow from the weighting agent blend (206) to be metered through the automated mud weight system (207). Accordingly, the HMI (201) monitors the output of the automated mud weight system (207) and terminates the flow of water (206c) when the specified volume of water has been dispensed from the weighting agent blend (206).

In Block 304, in response to a second control signal from the HMI (201), a specified amount of viscosifier (e.g., Bentonite) is added to the mixing tank (208). The specified amount is determined based on the viscosity specified by the user to the HMI (201). The second control signal actuates a second valve controlling the weighting agent blend (206) to dispense the Bentonite solution (206b) to be metered through the automated mud weight system (207) and the rig mixing pump (207b). Alternatively, the second control signal actuates an automated dispenser controlling the dry blend (204) to dispense the Bentonite powder to the mixing tank (208) via the rig mixing pump (207a). Concurrently, the viscosity of the mixture (i.e., water and Bentonite) in the mixing tank (208) is measured in real-time by the drilling fluid properties sensors (205). Accordingly, the HMI (201) monitors the output of the drilling fluid properties sensors (205) and deactivates the rig mixing pumps (207a) and (207b) when the specified viscosity has been reached. For example, the viscosity may be measure in terms of PV/YP. PV represents the viscosity of the drilling fluid when extrapolated to infinite shear rate based on the mathematical Bingham model. Yield point, YP, is the other parameter of the Bingham model. A low PV indicates that the drilling fluid is capable of drilling rapidly because of the low viscosity of drilling fluid exiting at the drill bit.

In Block 306, in response to a third control signal from the HMI (201), a specified amount of either dry blend mud (DBM, option #1 described below) or minimum additive mud (MAM, option #2 described below) are added in the mixing tank (208). The specified amount is determined based on the viscosity specified by the user to the HMI (201). The third control signal actuates a third valve controlling the weighting agent blend (206) to dispense the DBM or MAM in liquid form to be metered through the automated mud weight system (207) and the rig mixing pump (207b). Alternatively, the third control signal actuates an automated dispenser controlling the dry blend (204) to dispense the DBM or MAM in powder form to the mixing tank (208) via the rig mixing pump (207a).

Concurrently, the viscosity of the mixture (i.e., water and DBM or MAM) in the mixing tank (208) is measured in real-time by the drilling fluid properties sensors (205). Accordingly, the HMI (201) monitors the output of the drilling fluid properties sensors (205) and deactivates the rig mixing pumps (207a) and (207b) when the specified viscosity (e.g., measured as PV/YP) has been reached.

In Block 308, in response to a fourth control signal from the HMI (201), a specified amount of weighting agent (e.g. barite) is added in the mixing tank (208). The specified amount is determined based on the specific gravity specified by the user to the HMI (201). The fourth control signal actuates a fourth valve controlling the weighting agent blend (206) to dispense the barite in liquid form to be metered through the automated mud weight system (207) and the rig mixing pump (207b). Alternatively, the fourth control signal actuates an automated dispenser controlling the dry blend (204) to dispense the barite in powder form to the mixing tank (208) via the rig mixing pump (207a). Concurrently, the specific gravity of the mixture in the mixing tank (208) is measured in real-time by the drilling fluid properties sensors (205). Accordingly, the HMI (201) monitors the output of the drilling fluid properties sensors (205) and deactivates the rig mixing pumps (207a) and (207b) when the specified specific gravity has been reached.

In Block 310, in response to a fifth control signal from the HMI (201), a specified amount of pH buffer and optional additives (e.g., oxygen scavenger, sour gas scavenger, shale inhibiters, lubricant, etc.) is added in the mixing tank (208). The specified amount is determined based on various specified drilling fluid properties (e.g., pH value and rheological behavior) specified by the user to the HMI (201). The fifth control signal actuates a fifth valve controlling the weighting agent blend (206) to dispense the pH buffer and optional additives in liquid form to be metered through the automated mud weight system (207) and the rig mixing pump (207b). Alternatively, the fifth control signal actuates an automated dispenser controlling the dry blend (204) to dispense the pH buffer and optional additives in powder form to the mixing tank (208) via the rig mixing pump (207a). Concurrently, the pH value and/or rheological behavior of the mixture in the mixing tank (208) are measured in real-time by the drilling fluid properties sensors (205). Accordingly, the HMI (201) monitors the output of the drilling fluid properties sensors (205) and deactivates the rig mixing pumps (207a) and (207b) when the specified drilling fluid properties have been reached.

In Block 312, in response to a sixth control signal from the HMI (201), the final drilling fluid is released from the mixing tank (208) to circulate back through the drill string for continuous drilling.

As noted above, dry blend mud (DBM) is a physical blend of essential drilling fluid components (e.g., low temperature viscosifier, high temperature viscosifier, rheology modifier, filtrate loss control additive, and thinner) in appropriate ratio and capable of providing preliminary blend of wet blend mud (WBM) at densities from 8-22 ppg by adding water and Barite in required amounts. TABLE 1 lists an empirical relationship between amounts of DBM, water, and Barite to produce drilling fluid at densities from 8-22 ppg that is established in the laboratory to be used for automation.

TABLE 1
Mud density = f (m₁ (DBM), m₂ (Barite), m₃ (water)) where m₁, m₂, and m₃, are weights of DBM,
Barite and Water respectively.
S₁, S₂, S₃ are specific gravity of OSB, Barite and Water respectively.
V₁, V₂, V₃ are specific volume of OSB, Barite and Water respectively. Mud volume is calculated
from V = m/S
Total Mass of the mud blend M = Σm₁
Total Volume of the mud blend V = ΣV₁
Mud density = M/V

In the first stage mixing, water and Barite are mixed in required ratios to prepare DBM capable of providing preliminary blend of WBM at densities from 8-22 ppg. A density sensor is used at this stage to achieve the desired density of drilling fluid.

In the second stage mixing, the final WBM blend is prepared from the above preliminary blend of WBM by fine tuning the rheological parameters upon addition of controlled addition of liquid (diluted) version of various rheology modifiers and optional additives, such as pH Buffer, H₂S/CO₂ scavenger, oxygen scavenger, and shale inhibitor. A pH sensor, density sensor, and rheology sensor(s) are used to fine tune the corresponding drilling fluid parameters.

Further as noted above, minimum additive mud (MAM) is capable of providing preliminary blend of WBM at densities from 8-22 ppg by mixing water and Barite in required amounts. Table 2 lists an empirical relationship between MAM, water, and Barite amounts to produce drilling fluid at densities from 8-22 ppg that is established in the laboratory to be used for automation.

TABLE 2
Mud density = f (m₁ (MAM), m₂ (Barite), m₃ (water)) where
m₁, m₂, and m₃, are weights of MAM, Barite and Water respectively.
S₁, S₂, S₃ are specific gravity of MAM, Barite and Water respectively.
V₁, V₂, V₃ are specific volume of OSM, Barite and Water respectively. Mud volume is calculated
from V = m/S
Total Mass of the mud blend M = Σm₁
Total Volume of the mud blend V = ΣV₁
Mud density = M/V

In the first stage mixing, MAM, water, and Barite are mixed in required ratios to prepare DBM capable of providing preliminary blend of WBM at densities from 8-22 ppg. A density sensor is used at this stage to achieve the desired density of drilling fluid.

In the second stage mixing, the final WBM blend is prepared from the above preliminary blend of WBM by fine tuning the rheological parameters upon addition of controlled addition of liquid (diluted) version of various rheology modifiers and optional additives, such as pH Buffer, H₂S/CO₂ scavenger, oxygen scavenger, and shale inhibitor. A pH sensor, density sensor, and rheology sensor(s) are used to fine tune the corresponding drilling fluid parameters.

Embodiments may be implemented on a computing system. Any combination of mobile, desktop, server, router, switch, embedded device, or other types of hardware may be used. For example, as shown in FIG. 4A, the computing system (400) may include one or more computer processors (402), non-persistent storage (404) (e.g., volatile memory, such as random access memory (RAM), cache memory), persistent storage (406) (e.g., a hard disk, an optical drive such as a compact disk (CD) drive or digital versatile disk (DVD) drive, a flash memory, etc.), a communication interface (412) (e.g., Bluetooth interface, infrared interface, network interface, optical interface, etc.), and numerous other elements and functionalities.

The computer processor(s) (402) may be an integrated circuit for processing instructions. For example, the computer processor(s) may be one or more cores or micro-cores of a processor. The computing system (400) may also include one or more input devices (410), such as a touchscreen, keyboard, mouse, microphone, touchpad, electronic pen, or any other type of input device.

The communication interface (412) may include an integrated circuit for connecting the computing system (400) to a network (not shown) (e.g., a local area network (LAN), a wide area network (WAN) such as the Internet, mobile network, or any other type of network) and/or to another device, such as another computing device.

Further, the computing system (400) may include one or more output devices (408), such as a screen (e.g., a liquid crystal display (LCD), a plasma display, touchscreen, cathode ray tube (CRT) monitor, projector, or other display device), a printer, external storage, or any other output device. One or more of the output devices may be the same or different from the input device(s). The input and output device(s) may be locally or remotely connected to the computer processor(s) (402), non-persistent storage (404), and persistent storage (406). Many different types of computing systems exist, and the aforementioned input and output device(s) may take other forms.

Software instructions in the form of computer readable program code to perform embodiments of the disclosure may be stored, in whole or in part, temporarily or permanently, on a non-transitory computer readable medium such as a CD, DVD, storage device, a diskette, a tape, flash memory, physical memory, or any other computer readable storage medium. Specifically, the software instructions may correspond to computer readable program code that, when executed by a processor(s), is configured to perform one or more embodiments of the disclosure.

The computing system (400) in FIG. 4A may be connected to or be a part of a network. For example, as shown in FIG. 4B, the network (420) may include multiple nodes (e.g., node X (422), node Y (424)). Each node may correspond to a computing system, such as the computing system shown in FIG. 4A, or a group of nodes combined may correspond to the computing system shown in FIG. 4A. By way of an example, embodiments of the disclosure may be implemented on a node of a distributed system that is connected to other nodes. By way of another example, embodiments of the disclosure may be implemented on a distributed computing system having multiple nodes, where each portion of the disclosure may be located on a different node within the distributed computing system. Further, one or more elements of the aforementioned computing system (400) may be located at a remote location and connected to the other elements over a network.

Although not shown in FIG. 4B, the node may correspond to a blade in a server chassis that is connected to other nodes via a backplane. By way of another example, the node may correspond to a server in a data center. By way of another example, the node may correspond to a computer processor or micro-core of a computer processor with shared memory and/or resources.

The nodes (e.g., node X (422), node Y (424)) in the network (420) may be configured to provide services for a client device (426). For example, the nodes may be part of a cloud computing system. The nodes may include functionality to receive requests from the client device (426) and transmit responses to the client device (426). The client device (426) may be a computing system, such as the computing system shown in FIG. 4A. Further, the client device (426) may include and/or perform all or a portion of one or more embodiments of the disclosure.

While the disclosure has been described with respect to a limited number of embodiments, those skilled in the art, having benefit of this disclosure, will appreciate that other embodiments can be devised which do not depart from the scope of the disclosure as disclosed herein. Accordingly, the scope of the disclosure should be limited only by the attached claims.

Although the preceding description has been described herein with reference to particular means, materials and embodiments, it is not intended to be limited to the particulars disclosed herein; rather, it extends to all functionally equivalent structures, methods and uses, such as are within the scope of the appended claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112(1) for any limitations of any of the claims herein, except for those in which the claim expressly uses the words ‘means for’ together with an associated function.

What is claimed is:

1. A method for drilling fluid mixing and recycling, comprising:
receiving, by a human monitor interface (HMI) from a user, specified drilling fluid properties;
adding, in response to a first control signal from the HMI to control a first dispensing device, a volume of water in a mixing tank to reach a volume threshold;
adding, in response to a second control signal from the HMI to control a second dispensing device, a viscosifer in the mixing tank to reach a viscosity threshold;
adding, in response to a fourth control signal from the HMI to control a fourth dispensing device, a weighting agent in the mixing tank to reach a specific gravity threshold;
adding, in response to a fifth control signal from the HMI to control a fifth dispensing device, a pH buffer solution in the mixing tank to reach a pH threshold; and
releasing, in response to a sixth control signal from the HMI to control a sixth dispensing device, a final drilling fluid from the mixing tank to a drill string for continuous drilling,
wherein the volume threshold, the viscosity threshold, the specific gravity threshold, and the pH threshold are determined based on the specified drilling fluid properties.

2. The method of claim 1, further comprising:
receiving, by the HMI in real-time from a drilling fluid property sensor, a sensor output representing a water volume in the mixing tank; and
deactivating, by the HMI in response to the water volume in the mixing tank reaches the volume threshold, the first dispensing device.

3. The method of claim 1, further comprising:
receiving, by the HMI in real-time from a drilling fluid property sensor, a sensor output representing a viscosity of fluid in the mixing tank; and
deactivating, by the HMI in response to the viscosity reaching the viscosity threshold, the second dispensing device.

4. The method of claim 3, further comprising:
adding, in response to a third control signal from the HMI to control a third dispensing device, an amount of dry blend mud (DBM) or minimum additives mud (MAM) in the mixing tank to reach the viscosity threshold; and
deactivating, by the HMI in response to the viscosity reaching the viscosity threshold, the third dispensing device.

5. The method of claim 1, further comprising:
receiving, by the HMI in real-time from a drilling fluid property sensor, a sensor output representing a specific gravity of fluid in the mixing tank; and
deactivating, by the HMI in response to the specific gravity reaching the specific gravity threshold, the fourth dispensing device.

6. The method of claim 1, further comprising:
receiving, by the HMI in real-time from a drilling fluid property sensor, a sensor output representing a pH value of fluid in the mixing tank; and
deactivating, by the HMI in response to the pH value reaching the pH threshold, the fifth dispensing device.

7. The method of claim 1, further comprising:
adding, in response to a seventh control signal from the HMI to control a seventh dispensing device, rheological behavior additives in the mixing tank to reach a rheological behavior threshold;
receiving, by the HMI in real-time from a drilling fluid property sensor, a sensor output representing a measure of rheological behavior of fluid in the mixing tank; and
deactivating, by the HMI in response to the measure of rheological behavior reaching the rheological behavior threshold, the seventh dispensing device.

8. A system for drilling fluid mixing and recycling, comprising:
a plurality of real-time drilling fluid properties sensors configured to measure real-time fluid properties in a mixing tank;
a human monitor interface (HMI) configured to:
receiving a user input of specified drilling fluid properties;
receiving, from the plurality of real-time drilling fluid properties sensors; sensor outputs representing the real-time fluid properties in the mixing tank; and
generate, based on the user input and the sensor outputs, a sequence of control signals for drilling fluid mixing and recycling;
an automated material transfer unit configured to transfer chemical ingredients to an automated drilling fluid processing system; and
the automated drilling fluid processing system configured to:
add, in response to a first control signal from the HMI to control a first dispensing device, a volume of water in the mixing tank to reach a volume threshold;
add, in response to a second control signal from the HMI to control a second dispensing device, a viscosifer in the mixing tank to reach a viscosity threshold;
add, in response to a fourth control signal from the HMI to control a fourth dispensing device, a weighting agent in the mixing tank to reach a specific gravity threshold;
add, in response to a fifth control signal from the HMI to control a fifth dispensing device, a pH buffer solution in the mixing tank to reach a pH threshold; and
release, in response to a sixth control signal from the HMI to control a sixth dispensing device, a final drilling fluid from the mixing tank to a drill string for continuous drilling,
wherein the volume threshold, the viscosity threshold, the specific gravity threshold, and the pH threshold are determined based on the specified drilling fluid properties.

9. The system of claim 8, the HMI further configured to:
receive, from the plurality of real-time drilling fluid properties sensors, a sensor output representing a water volume in the mixing tank; and
deactivate, in response to the water volume in the mixing tank reaches the volume threshold, the first dispensing device.

10. The system of claim 8, the HMI further configured to:
receive, from the plurality of real-time drilling fluid properties sensors, a sensor output representing a viscosity of fluid in the mixing tank; and
deactivate, in response to the viscosity reaching the viscosity threshold, the second dispensing device.

11. The system of claim 10,
the automated drilling fluid processing system further configured to add, in response to a third control signal from the HMI to control a third dispensing device, an amount of dry blend mud (DBM) or minimum additives mud (MAM) in the mixing tank to reach the viscosity threshold; and
the HMI further configured to deactivate, in response to the viscosity reaching the viscosity threshold, the third dispensing device.

12. The system of claim 8, the HMI further configured to:
receive, from the plurality of real-time drilling fluid properties sensors, a sensor output representing a specific gravity of fluid in the mixing tank; and
deactivate, in response to the specific gravity reaching the specific gravity threshold, the fourth dispensing device.

13. The system of claim 8, the HMI further configured to:
receive, from the plurality of real-time drilling fluid properties sensors, a sensor output representing a pH value of fluid in the mixing tank; and
deactivate, in response to the pH value reaching the pH threshold, the fifth dispensing device.

14. The system of claim 8,
the automated drilling fluid processing system further configured to add, in response to a seventh control signal from the HMI to control a seventh dispensing device, rheological behavior additives in the mixing tank to reach a rheological behavior threshold;
the HMI further configured to:
receive, from plurality of real-time drilling fluid properties sensors, a sensor output representing a measure of rheological behavior of fluid in the mixing tank; and
deactivate, in response to the measure of rheological behavior reaching the rheological behavior threshold, the seventh dispensing device.

15. A non-transitory computer readable medium storing instructions executable by a computer processor for drilling fluid mixing and recycling, the instructions, when executed, comprising functionality for:
receiving, by a human monitor interface (HMI) from a user, specified drilling fluid properties;
adding, in response to a first control signal from the HMI to control a first dispensing device, a volume of water in a mixing tank to reach a volume threshold;
adding, in response to a second control signal from the HMI to control a second dispensing device, a viscosifer in the mixing tank to reach a viscosity threshold;
adding, in response to a fourth control signal from the HMI to control a fourth dispensing device, a weighting agent in the mixing tank to reach a specific gravity threshold;
adding, in response to a fifth control signal from the HMI to control a fifth dispensing device, a pH buffer solution in the mixing tank to reach a pH threshold; and
releasing, in response to a sixth control signal from the HMI to control a sixth dispensing device, a final drilling fluid from the mixing tank to a drill string for continuous drilling,
wherein the volume threshold, the viscosity threshold, the specific gravity threshold, and the pH threshold are determined based on the specified drilling fluid properties.

16. The non-transitory computer readable medium of claim 15, the instructions, when executed, further comprising functionality for:
receiving, by the HMI in real-time from a drilling fluid property sensor, a sensor output representing a water volume in the mixing tank; and
deactivating, by the HMI in response to the water volume in the mixing tank reaches the volume threshold, the first dispensing device.

17. The non-transitory computer readable medium of claim 15, the instructions, when executed, further comprising functionality for:
receiving, by the HMI in real-time from a drilling fluid property sensor, a sensor output representing a viscosity of fluid in the mixing tank; and
deactivating, by the HMI in response to the viscosity reaching the viscosity threshold, the second dispensing device.

18. The non-transitory computer readable medium of claim 17, the instructions, when executed, further comprising functionality for:
adding, in response to a third control signal from the HMI to control a third dispensing device, an amount of dry blend mud (DBM) or minimum additives mud (MAM) in the mixing tank to reach the viscosity threshold; and
deactivating, by the HMI in response to the viscosity reaching the viscosity threshold, the third dispensing device.

19. The non-transitory computer readable medium of claim 15, the instructions, when executed, further comprising functionality for:
receiving, by the HMI in real-time from a drilling fluid property sensor, a sensor output representing a specific gravity of fluid in the mixing tank; and
deactivating, by the HMI in response to the specific gravity reaching the specific gravity threshold, the fourth dispensing device.

20. The non-transitory computer readable medium of claim 15, the instructions, when executed, further comprising functionality for:
receiving, by the HMI in real-time from a drilling fluid property sensor, a sensor output representing a pH value of fluid in the mixing tank; and
deactivating, by the HMI in response to the pH value reaching the pH threshold, the fifth dispensing device.

21. The non-transitory computer readable medium of claim 15, the instructions, when executed, further comprising functionality for:
adding, in response to a seventh control signal from the HMI to control a seventh dispensing device, rheological behavior additives in the mixing tank to reach a rheological behavior threshold;
receiving, by the HMI in real-time from a drilling fluid property sensor, a sensor output representing a measure of rheological behavior of fluid in the mixing tank; and
deactivating, by the HMI in response to the measure of rheological behavior reaching the rheological behavior threshold, the seventh dispensing device.
